(19) United States
(12) Patent Application Publication
(10) Pub. No.: US 2026/0248383 A1
(43) Pub. Date: Aug. 27, 2026

(54) OPTOMETRIC EXAMINATION METHOD AND DEVICE THEREOF

(71) Applicant: Spectrovision Inc., Taipei City (TW)

(72) Inventors: JIH-YI LIAO, Taipei City (TW); SHIH-KAI LIN, Taipei City (TW); CHUNG-PING CHEN, Taipei City (TW); JIANN-HWA LUE, Taipei City (TW); CHIA-HUNG LIN, Taipei City (TW); MING-CHENG TSAI, Taipei City (TW); HUNG-CHIH YANG, Taipei City (TW); PIEN-PIEN LEE, Taipei City (TW); KUANG-CHAO HSU, Taipei City (TW)

(21) Appl. No.: 19/261,293

(22) Filed: Jul. 7, 2025

(30) Foreign Application Priority Data
Feb. 25, 2025 (TW) 114106900

(51) Int. Cl. A61B 3/11; A61B 3/00

(52) U.S. Cl. CPC A61B 3/11; A61B 3/0041

(57) ABSTRACT

An optometric examination method includes: using a collimator to capture a position of a pupil of the subject in real time, and calculating an objective pupil position reference information; using an optometric detector to emit an optometric examination light beam based on the objective pupil position reference information, calculate an ocular refraction information, and transmit the ocular refraction information to an examiner-end device; and using an augmented reality display to project a virtual examination visual target based on the objective pupil position reference information, and using a subject-end device to compare the virtual examination visual target with a subject response information input into a subject-end device, thereby obtaining an optometric result of the subject. The present disclosure integrates the augmented reality technology to achieve a highly accurate, efficient, interactive, and multifunctional optometric examination.

### BACKGROUND OF THE DISCLOSURE

### 1. Field of the Disclosure

The present disclosure relates to an examination method and device, and more particularly, to an optometric examination method and device.

### 2. Description of the Related Art

When light beam enters a human eye, it passes through the crystalline lens and is focused onto the macula of the retina, thereby producing a clear visual image. If the light is unable to be focused on the retina, the human eye perceives a blurred visual image. In such cases, the subject shall visit a clinic or hospital to have appropriate glasses prescribed in order to improve vision.

In current practice, an optometrist uses instruments to examine eye condition of the subject, such as determining diopter, in order to identify whether the subject exhibits myopia, hyperopia, or astigmatism.

However, the conventional optometric examination method primarily relies on subjective judgment of the optometrist to determine the examination results. If the optometrist lack sufficient experience or makes an incorrect judgment, the true eye conditions of the subject are not accurately determined. Moreover, the optometrist often requires multiple instruments to perform the complete optometric examination, causing low efficiency and poor cost-effectiveness.

In general, an optometric examination device capable of effectively performing optometric examinations is needed. Also, related technology incorporating augmented reality displays into the field of optometry has not yet been developed. Therefore, the industry is actively developing relevant technologies in an effort to provide technical solutions.

### SUMMARY OF THE DISCLOSURE

The present disclosure aims at providing an optometry examination method and device incorporating augmented reality, thereby assisting optometrists in performing eye examinations and improving the efficiency of optometry.

The aforementioned objectives do not prevent the existence of other objectives. Those objectives derivable from the specification, claims, or drawings of the present disclosure by a person having ordinary skill in the field of the invention are also included in the scope of objectives of the present disclosure.

For achieving the aforementioned objectives, the present disclosure provides an optometric examination method for conducting an optometric examination on a subject, comprising following steps:
using a collimator to capture a position of a pupil of the subject in real time, and calculating an objective pupil position reference information;
using an optometric detector to emit an optometric examination light beam based on the objective pupil position reference information, calculate an ocular refraction information, and transmit the ocular refraction information to an examiner-end device; and
using an augmented reality display to project a virtual examination visual target based on the objective pupil position reference information, and using a subject-end device to compare the virtual examination visual target with a subject response information input into a subject-end device, thereby obtaining an optometric result of the subject.

In another embodiment, the present disclosure provides an optometric examination device for conducting an optometric examination on a subject, comprising:
a trial frame comprising a trial lens, the trial lens being attachable to or detachable from the trial frame;
a collimator disposed on the trial frame, the collimator configured to capture a pupil position of a subject in real time and calculate an objective pupil position reference information;
an augmented reality display disposed on the trial frame, the augmented reality display configured to project a virtual examination visual target based on the objective pupil position reference information; and
a subject-end device coupled to the augmented reality display via a signal connection, the subject-end device configured to compare the virtual examination visual target with a subject response information input into the subject-end device, thereby obtaining an optometric result of the subject.

With such configuration, the present disclosure achieves following advantages.

The present disclosure includes the augmented reality (AR) technology to integrate various optometric examination devices, thereby eliminating the need for multiple different optometric devices and improving the efficiency of optometric examination.

The present disclosure prevents misjudgments that are possibly caused by subjective judgment or insufficient experience of the examiner.

The present disclosure captures the pupil position of the subject using a collimator, thereby ensuring the correctness of the light path and improving the precision of the examination.

The present disclosure adopts an interactive optometric examination method, not only improving the sense of engagement of the subject in the examination process, but also enabling the subject to respond based on actual visual perception to the virtual examination visual target, thereby improving the accuracy of the optometric result.

The present disclosure projects the virtual spatial visual target using the augmented reality display, and the calculation of ocular position information through the collimator, thereby further obtaining relative parameters of eyes, such as actual displacement, AC/A ratio, and CA/C ratio, thereby assisting the examiner in performing the optometric examination.

### BRIEF DESCRIPTION OF THE DRAWINGS

FIG. 1 is a perspective view of the optometric examination device in accordance with an embodiment of the present disclosure, particularly illustrating the appearance features of the optometric examination device.

FIG. 2 is a perspective view of the optometric examination device in accordance with another embodiment of the present disclosure.

FIG. 3 is a structural block view of the optometric examination device in accordance with another embodiment of the present disclosure, illustrating the arrangement relationships between each components.

FIG. 4 is an operational schematic view of the optometric examination device in accordance with another embodiment of the present disclosure, particularly illustrating the path of the light beam.

FIG. 5 is a flow chart of an optometric examination method in accordance with another embodiment of the present disclosure.

FIG. 6 is an operational schematic view of the collimator in accordance with another embodiment of the present disclosure, particularly illustrating the pupil center information.

FIG. 7 is another operational schematic view of the optometric examination device in accordance with an embodiment of the present disclosure, particularly illustrating the optometric detector collecting the optometric examination light beam.

FIG. 8 is another operational schematic view of the optometric examination device in accordance with an embodiment of the present disclosure, particularly illustrating the augmented reality display projecting the virtual spatial visual target.

FIG. 9 is another operational schematic view of the optometric examination device in accordance with an embodiment of the present disclosure, particularly illustrating the augmented reality display projecting the virtual spatial visual target.

FIG. 10 is a flow chart of an optometric examination method in accordance with another embodiment of the present disclosure.

FIG. 11 is another operational schematic view of the optometric examination device in accordance with an embodiment of the present disclosure, particularly illustrating the augmented reality display projecting the virtual graphical visual target.

### DETAILED DESCRIPTION OF THE DISCLOSURE

The aforementioned and further advantages and features of the present disclosure will be understood by reference to the description of the preferred embodiment in conjunction with the accompanying drawings where the components are illustrated based on a proportion for explanation but not subject to the actual component proportion.

Embodiments of the present disclosure are illustrated in detail along with the drawings. However, the technical features included by the present disclosure are not limited to certain embodiments hereby provided. Scope of the present disclosure shall be referred to the claims, which include all the possible replacements, modifications, and equivalent features.

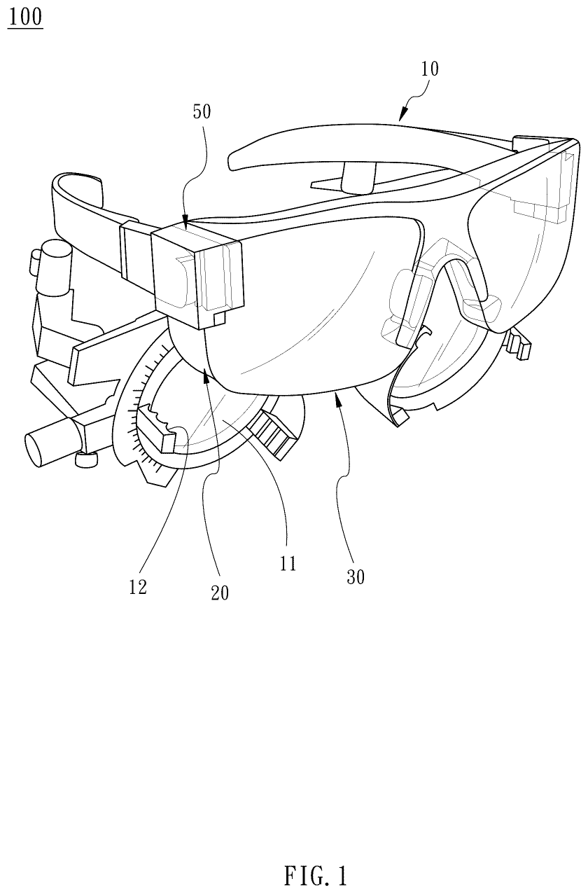

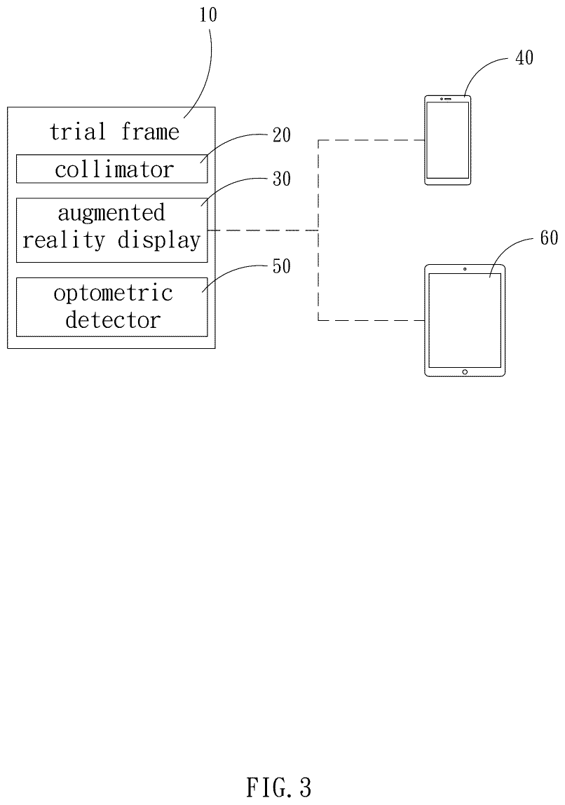

Referring to FIG. 1 to FIG. 7, in an embodiment, the present disclosure provides an optometric examination device 100 for conducting an optometric examination on a subject 4.

Referring to FIG. 1 to FIG. 3, the optometric examination device 100 comprises a trial frame 10, a collimator 20, an augmented reality display 30, and a subject-end device 40.

Referring to FIG. 1, the trial frame 10 is worn by the subject 4 and features lightweight and portability. The trial frame 10 has a trial lens 11, which is allowed to be a digital zoom lens, a physical zoom lens, a dual-prism zoom lens, or a lens of different diopters. The trial lens 11 is removably mounted on the trial frame 10.

In an embodiment, the trial frame 10 has at least one or more slot 12. The trial lens 11 is selectively disposed in the slot 12, allowing the optometrist to replace different trial lens 11. When the trial lens 11 is to be used, the trial lens 11 is allowed to be moved to a position on one side of the collimator 20 away from the augmented reality (AR) display 30 for performing the examination.

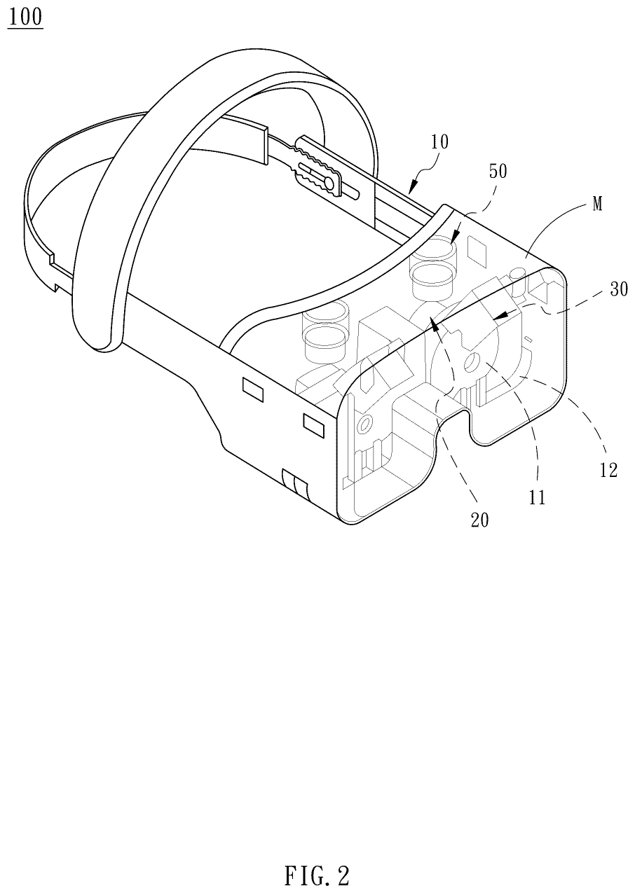

Referring to FIG. 2, the present disclosure provides another form of the optometric examination device 100 to improve the wearing comfort. In the embodiment, the optometric examination device 100 has a casing M combined with the trial frame 10. For ease of illustration, the casing M is shown in a transparent manner in FIG. 2 to reveal the components therein. The trial lens 11, the collimator 20, the augmented reality display 30, and the optometry detector 50 are disposed within the casing M, thereby improving the visual experience.

In another embodiment, the trial frame 10 comprises an X-axis adjustment device, a Y-axis adjustment device, and a Z-axis adjustment device so as to allow subject 4 to adjust the trial frame 10. Further, the X-axis adjustment device is configured to adjust the interpupillary distance, the Y-axis adjustment device is configured to adjust the position of the nose pad, and the Z-axis is configured to rotate the collimator 20.

The collimator 20 is disposed on the trial frame 10. Therein, the collimator 20 is capable of emitting an infrared light beam for performing calibration. The collimator 20 has an imaging device 21. The collimator 20 captures the eye of the subject 4 using the imaging device 21, so as to capture the pupil position of the subject 4 in real time and calculate an objective pupil position reference information.

The augmented reality display 30 is disposed on the trial frame 10. The augmented reality display 30 is configured to display various types of virtual visual targets, such as a virtual calibration visual target, a virtual examination visual target, a virtual spatial visual target 31, and a virtual graphical visual target, so as to provide the subject 4 with enhanced visual experiences. The augmented reality display 30 projects a virtual examination visual target based on the objective pupil position reference information. Therein, the virtual examination visual target is allowed to be patterns pointing upward, downward, leftward, rightward, or other examination patterns.

The subject-end device 40 is allowed to be a cellphone, tablet, or computer for the subject 4 to input a subject response information corresponding to the virtual examination visual target. The subject-end device 40 is coupled to the augmented reality display 30 via a signal connection, and configured to compare the virtual examination visual target with the subject response information input into the subject-end device 40, thereby obtaining an optometric result of the subject 4.

For example of the embodiment, the subject 4 wears the trial frame 10, and the augmented reality display 30 projects a virtual examination visual target. The subject 4 inputs the subject response information into the subject-end device 40 corresponding to the projected visual target. When the subject response information matches the virtual examination visual target, the augmented reality display projects another set of virtual examination visual targets, continuing the optometric examination process. When the subject response information does not match the virtual examination visual target, the subject-end device 40 accordingly obtains the optometric result of the subject 4, indicating that the trial lens 11 currently worn by the subject 4 needs to be adjusted. Therefore, with the iterative feedback from the aforementioned process, the optometric result of the subject 4 is optimized.

In an embodiment, the optometric examination device 100 comprises an optometric detector 50, which emits one or more light sources with stable wavelengths. The optometric detector 50 is disposed on the trial frame 10. The optometric detector 50 emits an optometric examination light beam based on the objective pupil position reference information, and calculates an ocular refraction information. By calculating using the objective pupil position reference information provided by the collimator 20, the error rate thereof is effectively reduced. In an embodiment, the optometric detector 50 calculates the refractive power, cylindrical power, and axis parameter using a built-in algorithm. The optometric detector 50 transmits the ocular refraction information to an examiner-end device 60, allowing the optometrist to obtain relevant information regarding the eyes of the subject.

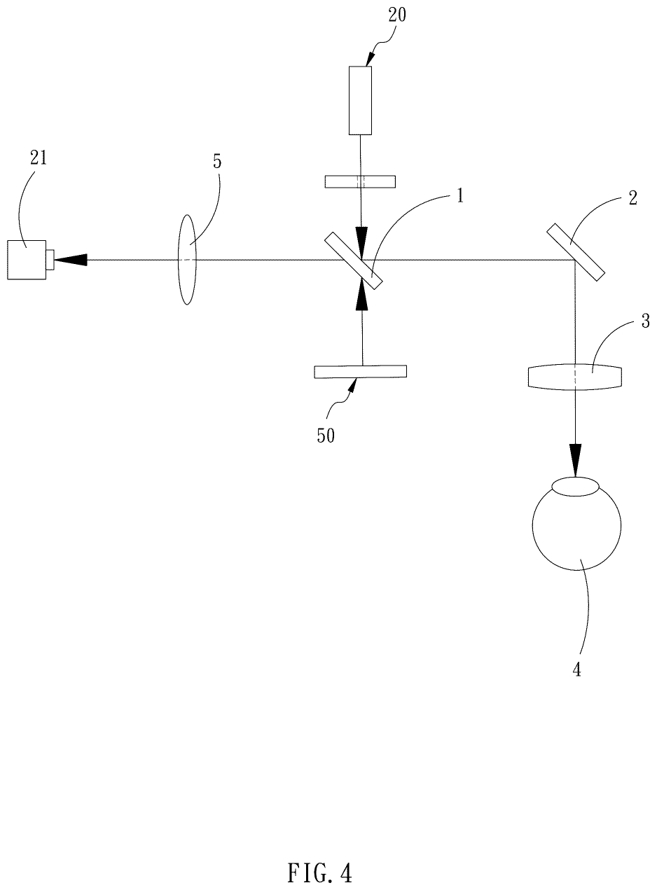

Referring to FIG. 4, the near-infrared light emitted by the optometric detector 50 sequentially passes through a beam splitter 1, a reflecting mirror 2, and a zoom lens 3, and then is focused onto the eye of the subject 4. The beam splitter 1 is configured to accurately separate multiple light sources and prevent the light sources from interfering the examination of eyes, wherein the beam splitter 1 has a splitting ratio of 90:10. The reflecting mirror 2 is configured to adjust the angle of the near-infrared light. The zoom lens 3 is configured to focus the near-infrared light, wherein the focal length of the zoom lens 3 ranges from 10 centimeters to 1 meter. Notably, the splitting ratio of the beam splitter 1 and the focal length of the zoom lens 3 are allowed to be adjusted according to actual requirements and are not limited thereto.

In an embodiment, the optometric detector 50 comprises an imaging device configured to capture the infrared light reflected from the eyes of the subject 4.

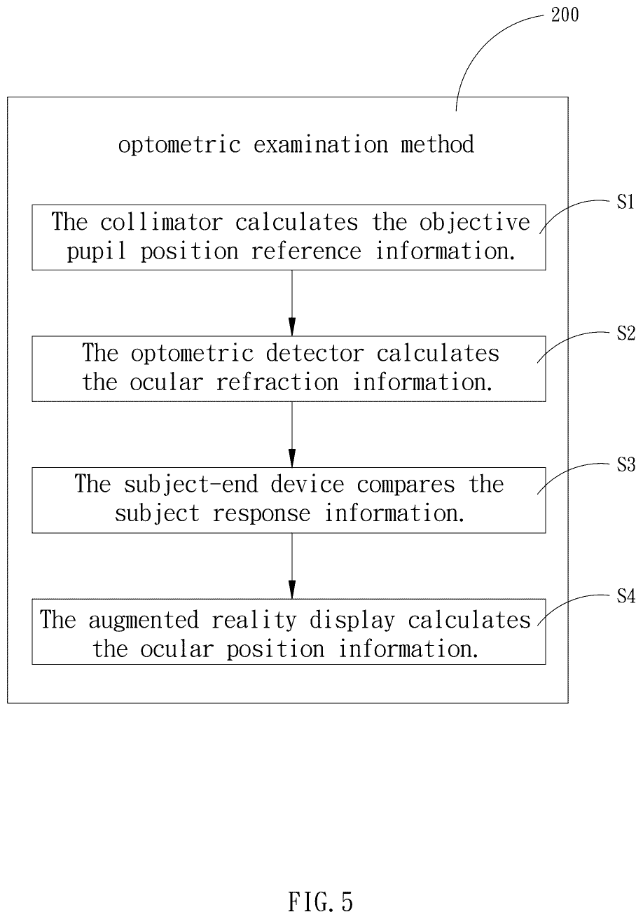

Referring to FIG. 5, the present disclosure provides a optometric examination method 200 for conducting an optometric examination on a subject 4. The optometric examination method 200 comprises following steps.

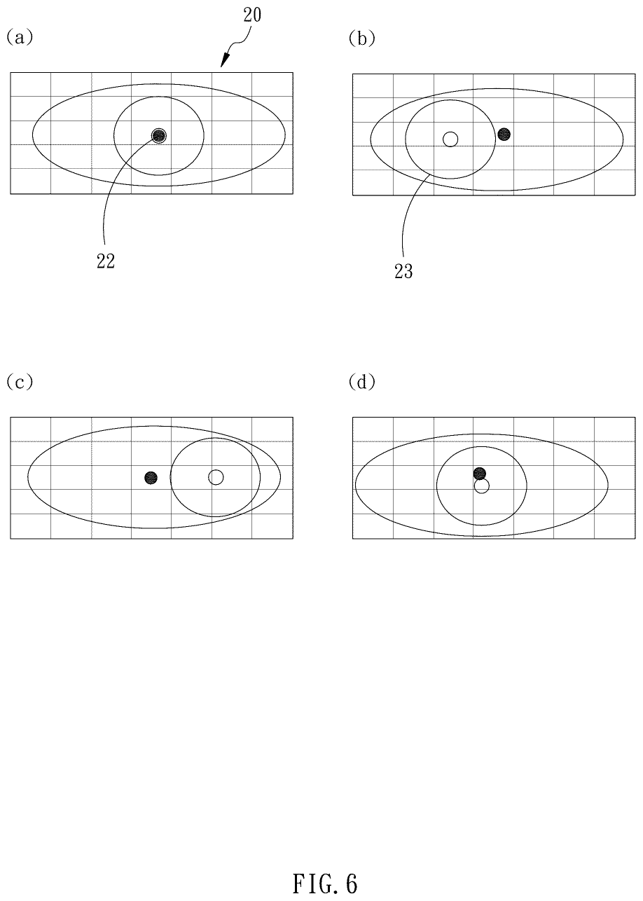

In step S1, a collimator 20 is used to capture the pupil position of a subject 4 and calculate an objective pupil position reference information. Referring to FIG. 6, the collimator 20 emits a calibration light beam, which is received by an eye of the subject 4, and the collimator 20 obtains a pupil center information 22. The collimator 20 calculates an objective pupil position reference information based on the pupil center information 22 and the pupil position.

The collimator 20 captures the pupil position of the subject 4 in real time to acquire multiple pupil images 23. Referring to FIG. 6(b) and FIG. 6(c), when the multiple pupil positions in the pupil images 23 do not match the pupil center information 22, the pupil images 23 are deemed unacceptable. Referring to FIG. 6(d), when the pupil position in at least one pupil image 23 matches the pupil center information 22, the corresponding pupil image 23 is deemed acceptable. The optometric detector 50 performs the calculation based on the acceptable pupil image 23 provided by the collimator 20, so as to reduce the error rate.

In step S2, an optometric detector 50 is used to emit an optometric light beam based on the objective pupil position reference information and calculate an ocular refraction information. The optometric detector 50 transmits the ocular refraction information to an examiner-end device 60.

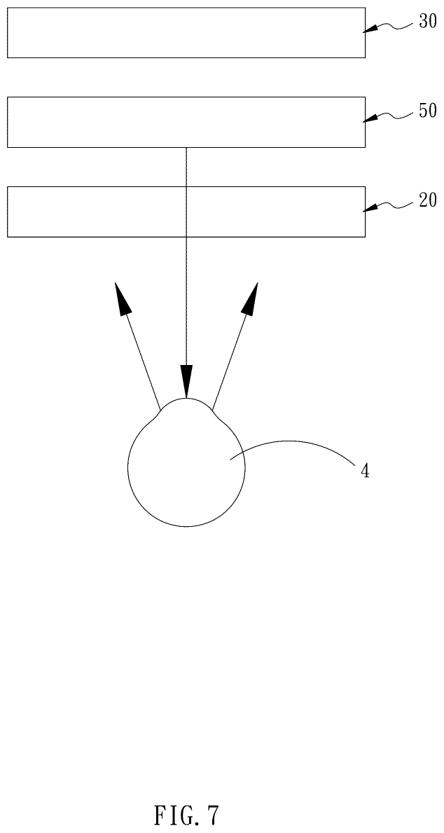

Referring to FIG. 7, the optometric detector 50 collects the optometric light beam reflected from the eyes of the subject 4 and calculates the ocular refraction information based on the optometric light beam. Specifically, the optometric detector 50 obtains a ocular refractive deviation information of the eye based on the deformation between the incident optometric light beam and the reflected optometric light beam. Then, the optometric detector 50 calculates the ocular refraction information using the refractive deviation information and a built-in algorithm.

In step S3, an augmented reality display 30 is used to project a virtual examination visual target based on the objective pupil position reference information. A subject-end device 40 is used to compare the virtual examination visual target with a subject response information input into the subject-end device 40, thereby obtaining an optometric result of the subject 4.

In step S4, the augmented reality display 30 is used to project a virtual spatial visual target 31 based on the object pupil position reference information and calculate an ocular position information. Then, the augmented reality display 30 transmits the ocular position information to the examiner-end device 60.

The augmented reality display 30 projects virtual spatial visual targets 31 toward the eyes of the subject 4 based on the objective pupil position reference information. The augmented reality display 30 calculates the ocular position information based on the distance at which the subject 4 gazes at the spatial visual targets 31.

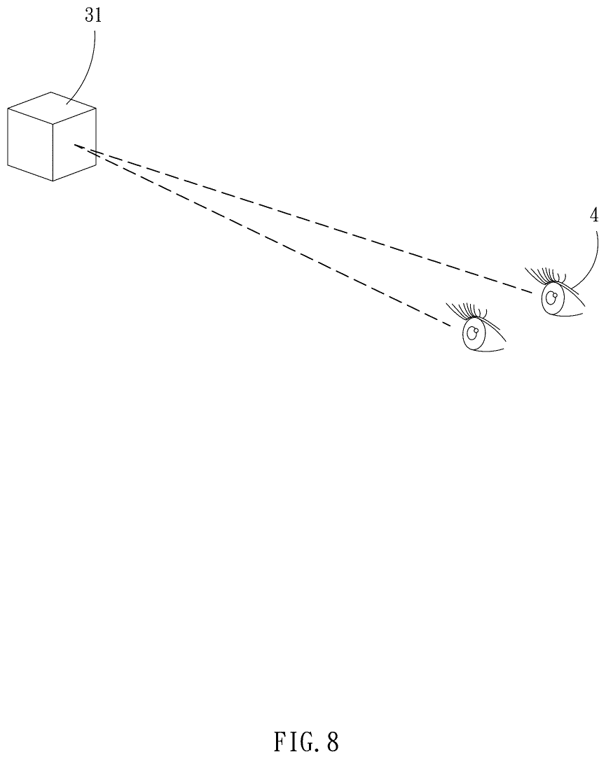

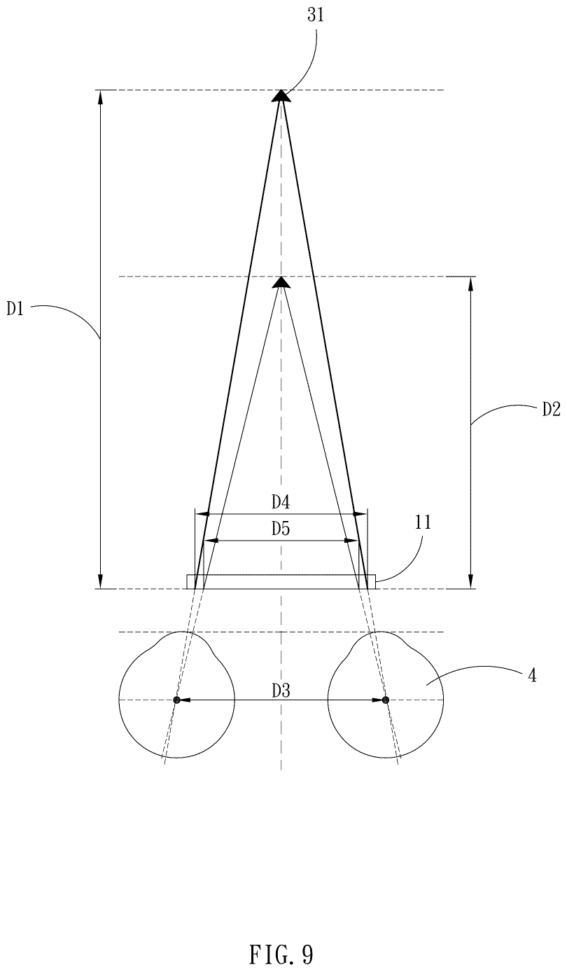

Referring to FIG. 8 and FIG. 9, the augmented reality display 30 projects different spatial visual targets 31 to two eyes, so that the subject 4 views the virtual visual targets at different distances. For example, the subject 4 is guided to view a virtual spatial visual target 31 at a first distance D1 and another virtual spatial visual target 31 at a second distance D2, so as to acquire the rotation angles of the eyes. The collimator 20 calculates an ocular convergence angle information based on the virtual spatial visual targets 31 viewed by the subject 4 at different distances. In an embodiment, the subject-end device 40 simultaneously determines an ocular convergence angle information based on the responses of the subject 4 to the virtual spatial visual targets at different distances, calculating the information related to the ocular position. Accordingly, the calculation accuracy is further improved.

Referring to FIG. 9, the first distance D1 is greater than the second distance D2. The distance between the two eyes is defined as the third distance D3. When the subject 4 views the virtual spatial visual target 31 at the first distance D1, the width of the line of sight of the subject 4 reaching the trial lens 11 is defined as the fourth distance D4. When the subject 4 views the virtual spatial visual target 31 at the second distance D2, the width of the line of sight of the subject 4 reaching the trial lens 11 is defined as the fifth distance D5.

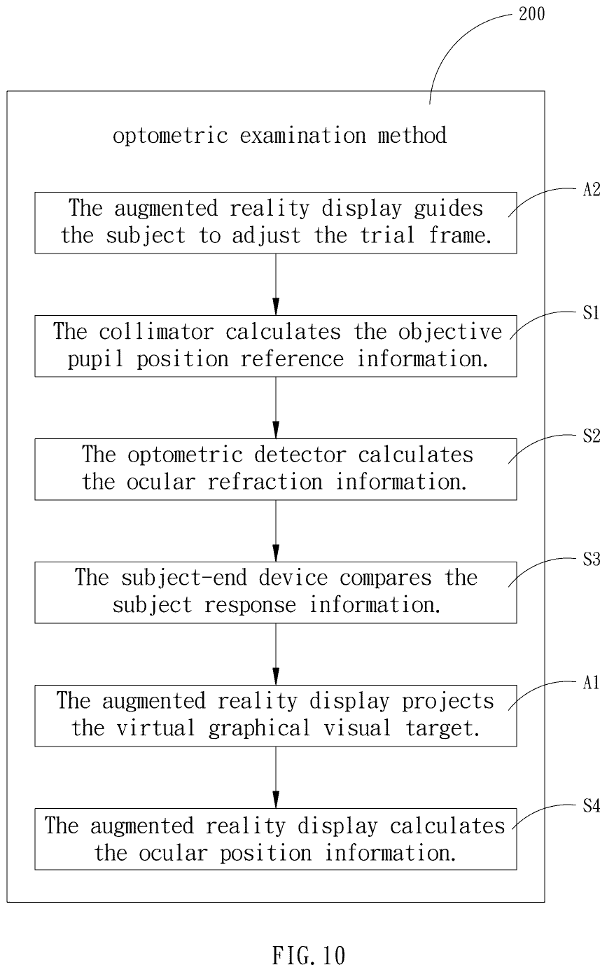

Referring to FIG. 10, a step A1 is performed after step S3 before step S4. In step A1, the augmented reality display 30 is used to project a virtual graphical visual target toward each eye of the subject 4 based on the objective pupil position reference information. The subject-end device 40 receives a dominant eye information corresponding to the virtual graphical visual target, so as to determine the dominant eye of the subject 4. Accordingly, the dominant eye information further improves the accuracy of convergence angle examination.

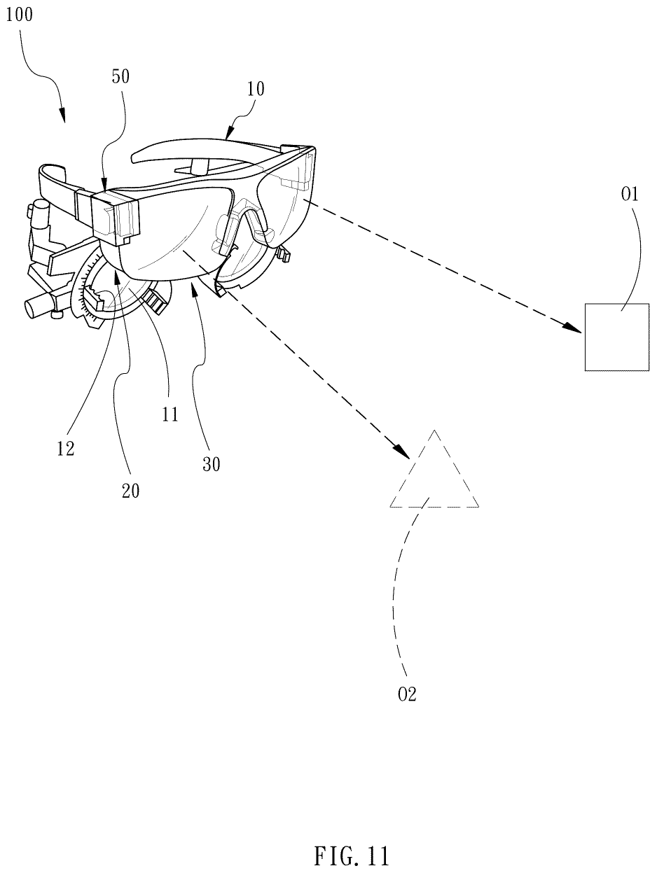

Referring to FIG. 11, the virtual graphical visual target comprises a test visual target O1 and a reference visual target O2. The test visual target O1 and a reference visual target O2 are viewed by the subject 4, so as to determine the dominant eye of the subject 4.

A step A2 is performed before step S1, wherein the trial frame 10 is adjusted in a subjective manner. In step A2, the augmented reality display 30 is used to project a virtual calibration visual target in order to guide the subject 4 to adjust the position of the trial frame 10, thereby positioning the trial frame 10 at the correct wearing position.

With the foregoing configuration, advantages of the present disclosure will be illustrated below.

The present disclosure includes the augmented reality technology to integrate various optometric examination devices, thereby eliminating the need for multiple different optometric devices and improving the efficiency of optometric examination.

The various examination devices in the present disclosure are capable of accurately determining ocular information, such as ocular diopter, AC/A ratio, and CA/C ratio, and providing such information to the optometrist. By doing so, the present disclosure helps avoid misjudgments caused by the subjective judgement or lack of experience of the optometrist.

The present disclosure captures the pupil position of the subject using a collimator 20, thereby ensuring the correctness of the light path and improving the precision of the examination.

The present disclosure not only identifies the pupil position using an objective method, but also allows the adjustment of the trial frame 10 in a subjective method. The combination of these two methods further improves the accuracy of the examination.

The present disclosure performs the optometric examination through an interactive process. Through iterative feedback between the augmented reality display 30 and the subject-end device 40, the optometric result of the subject 4 is optimized. The present disclosure not only improves the sense of engagement of the subject 4 in the examination process, but also enables the subject 4 to respond based on actual visual perception to the virtual examination visual target, thereby improving the accuracy of the optometric result.

The present disclosure uses the augmented reality display 30 to project the virtual spatial visual target 31, thereby obtaining relevant ocular parameters, capable of assisting the optometrist in performing optometric examination.

Although particular embodiments of the disclosure have been described in detail for purposes of illustration, various modifications and enhancements may be made without departing from the scope of the disclosure. Accordingly, the disclosure is not to be limited except as by the appended claims.

What is claimed is:

1. An optometric examination method for conducting an optometric examination on a subject, comprising following steps:
using a collimator to capture a position of a pupil of the subject in real time, and calculating an objective pupil position reference information;
using an optometric detector to emit an optometric examination light beam based on the objective pupil position reference information, calculate an ocular refraction information, and transmit the ocular refraction information to an examiner-end device; and
using an augmented reality display to project a virtual examination visual target based on the objective pupil position reference information, and using a subject-end device to compare the virtual examination visual target with a subject response information input into a subject-end device, thereby obtaining an optometric result of the subject.

2. The optometric examination method of claim 1, wherein the augmented reality display is configured to project a virtual spatial visual target based on the object pupil position reference information and calculate an ocular position information, and transmit the ocular position information to the examiner-end device.

3. The optometric examination method of claim 2, wherein the augmented reality display is configured to project a virtual calibration visual target to guide the subject to adjust a position of the trial frame.

4. The optometric examination method of claim 2, wherein the collimator is configured to emit a calibration light beam, which is received by an eye of the subject, and the collimator obtains a pupil center information; the collimator calculates the objective pupil position reference information based on the pupil center information and the pupil position.

5. The optometric examination method of claim 4, wherein the collimator is configured to capture the pupil position of the subject in real time to acquire multiple pupil images; when the pupil position in at least one of the pupil image matches the pupil center information, the corresponding pupil image is deemed acceptable and obtained; the collimator calculates the objective pupil position reference information based on the acceptable pupil image.

6. The optometric examination method of claim 2, wherein the optometric detector collects the optometric light beam reflected from the eye of the subject, and calculates the ocular refraction information based on the reflected optometric light beam.

7. The optometric examination method of claim 2, wherein the augmented reality display is configured to project the virtual spatial visual target to each eye of the subject, respectively, based on the objective pupil position reference information.

8. The optometric examination method of claim 7, wherein the collimator calculates the ocular position information based on a distance at which the subject gazes at the virtual spatial visual target.

9. The optometric examination method of claim 8, wherein the augmented reality display is configured to project a virtual graphical visual target toward each eye of the subject based on the objective pupil position reference information; the subject-end device receives a dominant eye information corresponding to the virtual graphical visual target.

10. An optometric examination device for conducting an optometric examination on a subject, comprising:
a trial frame comprising a trial lens, the trial lens being attachable to or detachable from the trial frame;
a collimator disposed on the trial frame, the collimator configured to capture a pupil position of a subject in real time and calculate an objective pupil position reference information;
an augmented reality display disposed on the trial frame, the augmented reality display configured to project a virtual examination visual target based on the objective pupil position reference information; and
a subject-end device coupled to the augmented reality display via a signal connection, the subject-end device configured to compare the virtual examination visual target with a subject response information input into the subject-end device, thereby obtaining an optometric result of the subject.

11. The optometric examination device of claim 10, wherein the optometric examination device comprises an optometric detector; the optometric detector is disposed on the trial frame; the optometric detector is configured to emit an optometric examination light beam based on the objective pupil position reference information, calculate an ocular refraction information, and transmit the ocular refraction information to an examiner-end device.

12. The optometric examination device of claim 11, wherein the trial lens is selected from a group consisting of a digital zoom lens, a physical zoom lens, and a dual-prism zoom lens.
